(19) United States
(12) Patent Application Publication
(10) Pub. No.: US 2013/0096363 A1
(43) Pub. Date: Apr. 18, 2013

(54) NEUROMODULATION OF DEEP-BRAIN TARGETS BY TRANSCRANIAL MAGNETIC STIMULATION ENHANCED BY TRANSCRANIAL DIRECT CURRENT STIMULATION

(71) Applicants: M. Bret Schneider, Portola Valley, CA (US); Michael J. Partsch, Redwood City, CA (US); David J. Mishelevich, Playa del Rey, CA (US)

(72) Inventors: M. Bret Schneider, Portola Valley, CA (US); Michael J. Partsch, Redwood City, CA (US); David J. Mishelevich, Playa del Rey, CA (US)

(21) Appl. No.: 13/636,022

(22) PCT Filed: Mar. 30, 2011
(86) PCT No.: PCT/US11/30550

§ 371 Date: Dec. 21, 2012

Related U.S. Application Data
(60) Provisional application No. 61/320,467, filed on Apr. 2, 2010.

(51) Int. Cl. A61N 2/02

(52) U.S. Cl. CPC A61N 2/02
USPC 600/13

(57) ABSTRACT

Described herein are methods, devices and systems for neuromodulation of deep brain targets using a combination of transcranial magnetic stimulation (TMS) and transcranial direct current (DC) stimulation to reduce or eliminate side-effects when modulating one or more deep brain targets. For example, transcranial magnetic stimulation of a deep brain target may be synchronized with modulation of more superficially located cortical brain regions using transcranial direct current stimulation to prevent seizures and other side effects. Systems configured to regulate (or synchronize) the application of transcranial magnetic stimulation of deep brain targets and transcranial direct current stimulation are also described.

### CROSS REFERENCE TO RELATED APPLICATIONS

This patent application may be related to one or more of the following patents and pending patent applications (US and PCT applications), each of which is herein incorporated by reference in its entirety: U.S. Pat. No. 7,520,848, titled “ROBOTIC APPARATUS FOR TARGETING AND PRODUCING DEEP, FOCUSED TRANSCRANIAL MAGENTIC STIMULATON”, issued on Apr. 21, 2009; U.S. patent application Ser. No. 12/402,404, titled “ROBOTIC APPARATUS FOR TARGETING AND PRODUCING DEEP, FOCUSED TRANSCRANIAL MAGENTIC STIMULATON”, filed on Mar. 11, 2009; U.S. patent application Ser. No. 11/429,504, titled “TRAJECTORY-BASED DEEP-BRAIN STEREOTACTIC TRANSCRANIAL MAGNETIC STIMULATION”, filed on May 5, 2006; U.S. patent application Ser. No. 12/669,882, titled “DEVICE AND METHOD FOR TREATING HYPERTENSION VIA NON-INVASIVE NEUROMODULATION”, filed on Jan. 20, 2010; U.S. patent application Ser. No. 12/671,260, titled “GANTRY AND SWITCHES FOR POSITION-BASED TRIGGERING OF TMS PULSES IN MOVING COILS”, filed on Jan. 29, 2010; U.S. patent application Ser. No. 12/670,938, titled “FIRING PATTERNS FOR DEEP BRAIN TRANSCRANIAL MAGNETIC STIMULATION”, filed on Jan. 27, 2010; U.S. patent application Ser. No. 12/677,220, titled “FOCUSED MAGNETIC FIELDS”, filed on Mar. 9, 2010; PCT Application No. PCT/US2008/077851, titled “SYSTEMS AND METHODS FOR COOLING ELECTROMAGNETS FOR TRANSCRANIAL MAGNETIC STIMULATION”, filed on Sep. 26, 2008; PCT Application No. PCT/US2008/081048, titled “INTRA-SESSION CONTROL OF TRANSCRANIAL MAGNETIC STIMULATION”, filed on Oct. 24, 2008; U.S. patent application Ser. No. 12/324,227, titled “TRANSCRANIAL MAGNETIC STIMULATION OF DEEP BRAIN TARGETS”, filed on Nov. 26, 2008; PCT Application No. PCT/US2009/045109, titled “TRANSCRANIAL MAGNETIC STIMULATION BY ENHANCEDMAGNETIC FIELD PERTURBATIONS”, filed on May 26, 2009; U.S. patent application Ser. No. 12/185,544, titled “MONOPHASIC MULTI-COIL ARRAYS FOR TRANSCRANIAL MAGNETIC STIMULATION”, filed on Aug. 4, 2008; U.S. patent application Ser. No. 12/701,395, titled “CONTROL AND COORDINATION OF TRANSCRANIAL MAGNETIC STIMULATION ELECTROMAGNETS FOR MODULATION OF DEEP BRAIN TARGETS”, filed on Feb. 5, 2010; and PCT Application No. PCT/US2010/020324, titled “SHAPED COILS FOR TRANSCRANIAL MAGNETIC STIMULATION”, filed on Jan. 7, 2010.

### INCORPORATION BY REFERENCE

All publications, including patents and patent applications, mentioned in this specification are herein incorporated by reference in their entirety to the same extent as if each individual publication was specifically and individually indicated to be incorporated by reference.

### FIELD OF THE INVENTION

Described herein are systems and methods for deep brain transcranial magnetic stimulation while suppressing or reducing side effects such as seizures. In particular, described herein are devices, systems and method for deep-brain stimulation using combined transcranial magnetic stimulation (TMS) and transcranial direct current stimulation (tDCS).

### BACKGROUND OF THE INVENTION

Neuromodulation of superficial cortex of the brain by Transcranial Magnetic Stimulation (TMS) was first described by Barker et al. (“Non-invasive magnetic stimulation of human motor cortex,” Lancet. 1985; 1(8437):1106-1107) and has been traditionally been done with one “double coil” electromagnet powered by single electromagnetic pulse source.

Transcranial Magnetic Stimulation (TMS) of cortical regions has been demonstrated to have positive clinical results for indications, such as depression (O'Reardon, J. P., Solvason, H. B., Janicak, P. G., et al., “Efficacy and safety of transcranial magnetic stimulation in the acute treatment of major depression: a multisite randomized controlled trial,” Biol Psychiatry. 2007 Dec. 1;62:1208-1216. Epub 2007 Jun. 14 and Fitzgerald, P. B., Brown, T. L., Marston, N. A., et al., “Transcranial magnetic stimulation in the treatment of depression: a double-blind, placebo-controlled trial,” Arch Gen Psychiatry. 2003;60:1002-1008), Post-Traumatic Stress Disorder (PTSD) (Cohen, H, Kaplan, Z., Kotler, M., et al., “Repetitive transcranial magnetic stimulation of the right dorsolateral prefrontal cortex in posttraumatic stress disorder: a double-blind, placebo-controlled study,” Am J Psychiatry. 2004;161:515-524), Parkinson's Disease (Khedr, E. M., Farweez, H. M., and H. Islam, Therapeutic effect of repetitive transcranial magnetic stimulation on motor function in Parkinson's disease patients,” Eur. J Neurol. 2003;10:567-572), tinnitus (Kleinjung, T., Vielsmeier, V., Landgrebe, M., Hajak, G. and B., Langguth, “Transcranial magnetic stimulation: a new diagnostic and therapeutic tool for tinnitus patients”. Int Tinnitus J 14 (2): 112-8, 2008), stroke (Stroke (Mansur, C. G., Fregni, F., Boggio, P. S., Riberto, M., Gallucci-Neto, J., Santos, C. M., Wagner, T., Rigonatti, S. P., Marcolin, M. A., Pascual-Leone, A., “A sham stimulation-controlled trial of rTMS of the unaffected hemisphere in stroke patients,” Neurology 2005, 64:1802-1804), schizophrenia (Aleman, A., Sommer, I. E., Kahn, R. S., “Efficacy of slow repetitive transcranial magnetic stimulation in the treatment of resistant auditory hallucinations in schizophrenia: a meta-analysis,” J Clin Psychiatry. 2007 March;68(3):416-21), amyotrophic lateral sclerosis (Zanette, G., Forgione, A., Manganotti, P., et al., “The effect of repetitive transcranial magnetic stimulation on motor performance, fatigue and quality of life in amyotrophic lateral sclerosis,” J Neurol Sci. 2008 Feb. 26), obsessive compulsive disorder (OCD) (Alonso, P., Pujol, J., Cardoner, N., et al., “Right prefrontal repetitive transcranial magnetic stimulation in obsessive-compulsive disorder: a double-blind, placebo-controlled study,” Am J Psychiatry. 2001;158:1143-1145), pain (Andre-Obadia, N., Mertens, P., Gueguen, A., et al., “Pain relief by rTMS: differential effect of current flow but no specific action on pain subtypes,” Neurology. 2008;71:833-840), and seizures (Theodore, W. H., Hunter, K., Chen, R., et al., ‘Transcranial magnetic stimulation for the treatment of seizures: A controlled study,” Neurology. 2002;59:560-562).

Stimulation of deep brain target regions using traditional Transcranial Magnetic Stimulation typically requires an increased level of stimulation because the magnetic flux falls off as a function of distance according to known principles. The attenuation of the magnetic field is known to be proportional to 1/(distance)² at short distances This inverse-square relationship is particularly significant, and a version of this relationship has been used to determine the strength needed for stimulation of a deep brain target region by one or more TMS electromagnets. Thus to reach deep neural structures using systems that are designed for superficial TMS (normally using a single electromagnet) will require turning up the stimulation intensity. Unfortunately this may contribute to overstimulation of the superficial cortex (including possibly seizures) when the cortex or is closer to the TMS electromagnet than the intended target.

Recently we have developed systems and techniques for neuromodulation of deep-brain targets previously believed to be inaccessible to TMS. Modulation of deep-brain regions at helpful magnetic field strengths was believed impossible because the drop in field strength with depth would require an extremely high magnetic field at more cortical regions in order to reach the deep brain regions. However, the technique for focused deep-brain neuromodulation we have developed (e.g., U.S. Pat. No. 7,520,848, and application PCT/US2007/010262) permit stimulation of deep brain regions without over-stimulating intervening cortical regions of the patient's brain.

Other groups have also developed techniques for stimulation of deep-brain regions, however with substantially less specificity and focus. For example, a Hesed Coil (U.S. Pat. No. 7,407,478) has been proposed for deep-brain neuromodulation by applying a magnetic field through the entire brain in such a manner that the field falls off more slowly than with standard TMS electromagnets. However, this technique still stimulates the deep-brain regions less than more cortical regions, and the applied field is unfocused.

The undesirable consequence of stimulation of more cortical brain regions even when targeting deep brain regions may include side effects such as pain, dizziness, nausea, and even seizures. Thus, it would be beneficial when stimulating deep brain target regions to reduce the side effects that may arise when stimulating more cortical regions. Described herein are methods and devices for stimulating of deep brain targets while suppressing or reducing side effects (including seizures) even at relatively large field strengths, by the specific application of transcranial direct current stimulation (tDCS).

Although the devices, systems and methods described herein are discussed in the context of deep brain modulation (and may find special applicability with deep brain stimulation), these techniques may be generalizable to standard TMS and other variations of TMS. Examples of typical TMS systems may be found in the literature (e.g., TMS has been used in conjunction with both EEG and imaging). Pulsed stimulators for Transcranial Magnetic Stimulation are provided by multiple vendors (e.g., Magstim in the U.K. with the Rapid² and related devices and MagVenture in Denmark with its MagPro series of stimulators).

Transcranial direct current stimulation (tDCS) has been described a method for neuromodulation of the superficial cortex of the brain. The application of weak static (DC) electrical currents (on the order of 1 to 2 mA) to the scalp is believed to cause neuronal membranes to either partially depolarize, in which case the firing rate increases, or to partially hyperpolarized, in which the firing rate decreases. Partial depolarization occurs when the neuron is near the positive electrode (the anode) and partial hyperpolarization occurs when the neuron is near the negative electrode (the cathode). Since the tDCS electrodes are located on the scalp of the patient with skin, subcutaneous structures, skull, and brain coverings between the electrodes and the cortex the current density applied is very small. Partial depolarization or hyperpolarization is small, on the order of a fraction of a millivolt. Voltage may be applied up to 10 volts (V). Above 10 V, there will be a scalp sensation which may be uncomfortable. The effect of the application of tDCS persists after the tDCS is no longer present, ranging from a few minutes to over an hour.

An example of a tDCS instrument is the Magstim eldith (produced by the neuroConn company in Germany) DC-STIMULATOR (and DC-STIMULATOR-Plus) producing biphasic pulses up to 3 mA peak-to-peak at single frequencies of up to 250 Hz, up to 30 minutes. Like TMS, tDCS has been used in conjunction with both EEG and imaging.

The size of the electrodes for tDCS usually is in the range of 5 cm² to 50 cm², say sponges of 4 cm by 6 cm. It is desirable to maximize electrode separation to decrease the quantity of the current flowing over the scalp as opposed to going through the brain. Pad-style electrodes are provided by Magstim. Other electrode designs are applicable such as a paddle format with electrodes housed in an enclosure with the electrical path being via a fluid passage (B. Simon, “Methods and Apparatus for Transcranial Stimulation,” U.S. Patent Application US2009/0319002, Dec. 24, 2009).

The transcranial Direct Current Stimulation (tDCS) has been demonstrated to have positive clinical results for indications such as fibromyalgia (Roizenblatt, S., Fregni, F., Gimenez, R., Wetzel, T., Rigonatti, S. P., Tufik, S., Boggio, P. S., and A. C. Valle, “Site-specific Effects of Transcranial Direct Current Stimulation on Sleep and Pain in Fibromyalgia: A Randomized, Sham-controlled Study,” Pain Practice, Volume 7, Issue 4, 2007 297-306), depression (Bikson, M., Bulow, P., Stiller,, J. W., Datta, A., Fortunato, M. S., Battaglia, F., Karnup, S. V., and T. T. Postolache, “Transcranial Direct Current Transcranial Direct Current Stimulation for Major Depression: A General System for Quantifying Transcranial Electrotherapy Dosage Transcranial Electrotherapy Dosage,” Current Treatment Options in Neurology, 10:377-385, 2008 and Boggioa, P. S., Rigonatti, S. P., Ribeiro, R. B., Myczkowski, M. L., Nitsche, M. A., Pascual-Leone, A., and F. Fregnia, “A randomized, double-blind clinical trial on the efficacy of cortical direct current stimulation for the treatment of major depression,” The International Journal of Neuropsychopharmacology, 11:249-254, 2008), pain perception (Antal, A., Brepohl, N., Poreisz, C., Boros, K., Csifcsak, G., and W. Paulus, “Transcranial direct current stimulation over somatosensory cortex decreases experimentally induced acute pain perception,” Clin. J Pain., January;24(1):56-63, 2008),

tDCS can cause other physiologic changes as well such as enhancement of working memory (Fregni, F., Boggio, P. S., Nitsche, M., Bermpohl, F., Antal, A., Feredoes, E., Marcolin, M. A., Rigonatti, S. P., Silva, M. T., Paulus, W., and A. Pascual-Leone, “Anodal transcranial direct current stimulation of prefrontal cortex enhances working memory,” Exp. Brain Res., 2005 September;166(1):23-30. Epub 2005 Jul. 6), improvement in spatial tactile acuity (Ragert, P., Vandermeeren, Y., Camus, M., and L. G. Cohen, “Improvement of spatial tactile acuity by transcranial direct current stimulation,” Clin Neurophysiol,. 2008 April;119(4):805-11, 2008 Epub 2008 Jan. 18), enhancement of language performance (Sparing, R., Dafotakis, M., Meister, I. G., Thirugnanasambandam, N., and G. R. Fink, “Enhancing language performance with non-invasive brain stimulation—a transcranial direct current stimulation study in healthy humans,” Neuropsychologia. 2008 Jan. 15;46(1):261-8. Epub 2007 Jul. 24), and decrease in risk-taking behavior (Fecteau, S., Knoch, D., Fregni, F., Sultani, N., Boggio, P., and A. Pascual-Leone, “Diminishing risk-taking behavior by modulating activity in the prefrontal cortex: a direct current stimulation study,” J. Neurosci. 2007 Nov. 14;27(46):12500-5).

TDCS effectively only reaches the cortical surface of the brain, and not to elements of the brain which are not in contact with the subdural pool of cerebral spinal fluid. This is because the spread of electrical current depends upon this energy form passing through highly conductive media. Conductivity in the cerebral spinal fluid is about 1.654 S/m, in the gray manner is 0.276 S/m. Conductivity in underlying in a direction parallel to nerve tracts is 1 S/nm, while conductivity perpendicular to tracts is 0.1 S/m.

Although tDCS has been used in conjunction with TMS, the two techniques have been applied only to cortical brain regions. For example, tDCS has been proposed as a way to “precondition” motor or visual cortex to prepare it for later TMS stimulation. In one early study, Nitsche and Paulus (Journal of Clinical Physiology, 527, 3:633-639, 2000), used tDCS to examine the change in the motor-evoked potential as determined by TMS. The effect was durable in that it was remained for a few minutes after the tDCS stimulus was stopped. The amplitude and length of the effect was found to be related to the intensity of the current and the duration of stimulation. It has also been shown that anodal polarization of the motor cortex increased motor responsiveness to TMS stimulation and that cathodal polarization decreased such responsiveness. One study did this in the context of sensitizing the motor cortex to TMS stimulation by applying preconditioning with tDCS (Lang, N., Siebner, H. R., Ernst, D., Nitsche, M. A., Paulus, P., Lemon, R. N., and J. C. Rothwell, “Preconditioning with transcranial direct current stimulation sensitizes the motor cortex to rapid-rate transcranial magnetic stimulation and controls the direction of after-effects,” Biological Psychiatry, Volume 56, Issue 9, 1 Nov. 2004, Pages 634-639). Thus, in the motor cortex, tDCS can influence excitability changes demonstrated by applying rTMS. This was also shown in the visual cortex, but to a lesser degree (Lang, N., Siebner, H. R., Boros, K., Nitsche, M. A., Paulus, W., A. Antal, and J. C. Rothwell, “Bidirectional Modulation of Primary Visual Cortex Excitability: A Combined tDCS and rTMS Study,” Investigative Ophthalmology and Visual Science, 2007;48:5782-5787., oi:10.1167/iovs.07-0706).

To date, these studies combining tDCS and TMS have been applied only to cortical brain regions, in which tDCS was applied primarily to sensitize the same cortical regions to later (subsequently applied) TMS. These studies have also strongly suggested that the cortical location to which tDCS is applied (e.g., motor, visual, etc.) is critical to understanding the effect on excitability of the cortical region. Finally, these studies have relied upon the sequential (e.g. tDCS followed by TMS) rather than concurrent or simultaneous application of tDCS and TMS.

Riehl et al. (U.S. Pat. No. 7,153,256) describe a system of electrical stimulating applied to a subject's head to reduce the pain of TMS that shares some features of tDCS, but is not tDCS. In Riehl, superficial nerve/muscle stimulation on the scalp is applied in conjunction with TMS to provide a distraction and reduce sensation from the magnetic field caused by TMS stimulating scalp structures. However, the stimulation applied by Riehl is not tDCS; tDCS stimulates the cortex, and is not limited to the superficial tissue (e.g., skin, muscle, etc.). Further, Riehl applies varying current, resulting in an induced magnetic field that, in some variations, is matched to the applied TMS field. Thus Riehl does not apply direct current. Riehl is intended to reduce patient discomfort in a way that would not stabilize superficial cortex to prevent seizures.

As described above, it would be desirable not just to stimulating deep brain regions using TMS in conjunction with a variation of tDCS that reduces or inhibits side effects, and particularly seizures to enhance the safety and efficacy of neuromodulation.

### SUMMARY OF THE INVENTION

The methods, systems and devices described herein may be used for transcranial magnetic stimulation (TMS) of deep brain targets while reducing or eliminating side effects such as seizures by modulating cortical brain regions using direct current (e.g., transcranial direct current stimulation or tDCS). Deep brain TMS may be applied by one or more moving TMS electromagnets, or by an array of TMS electromagnets, where the emitted electromagnetic field is focused on the deep brain target. More superficially located brain regions (e.g., cortical brain regions) between the TMS electromagnet and the deep brain target, are modulated by the application of an appropriate tDCS (anodal or cathodal). The position of the TMS electromagnet(s) may be coordinated with the application of the anodal or cathodal tDCS. In this manner, TMS neuromodulation may be applied to deep brain targets while superficial cortical structures are stabilized by tDCS to reduce or eliminate the chance that seizure or other side effects.

For example, described herein are methods for applying transcranial magnetic stimulation to modulate a deep brain target while reducing side effects by modulating cortical brain regions, the method comprising: targeting a deep-brain target region with one or more transcranial magnetic stimulation electromagnets; applying transcranial direct current stimulation from the scalp to modulate excitability of cortical brain regions between the one or more transcranial magnetic stimulation electromagnets and the deep-brain target; and applying transcranial magnetic stimulation to the deep brain target while modulating the cortical regions by the transcranial direct current stimulation.

The step of applying transcranial magnetic stimulation may comprise aiming a plurality of transcranial magnetic stimulation electromagnets at the deep brain target. Alternatively, the step of applying transcranial magnetic stimulation may comprise aiming at least one transcranial magnetic stimulation electromagnet at the deep brain target while moving the transcranial magnetic stimulation electromagnet about the subject's head.

In some variations, the step of applying transcranial magnetic stimulation to the deep brain target comprises applying energy to the transcranial magnetic stimulation electromagnets while concurrently applying the transcranial direct current stimulation from the scalp. For example, the steps of applying transcranial direct current stimulation and applying transcranial magnetic stimulation are simultaneously applied. The transcranial magnetic stimulation may be applied at the same time that the transcranial direct current stimulation is applied to the superficial cortical brain region(s), or at any time while the appropriate superficial cortical brain regions are modulated by the transcranial direct current stimulation, including shortly (or immediately) after termination of the transcranial direct current stimulation. As mentioned above, the effect of transcranial direct current stimulation on cortical regions may last for minutes after the application of direct current has stopped.

The step of applying transcranial direct current stimulation may comprise applying either (or both) cathodal or anodal transcranial direct current stimulation.

The transcranial direct current stimulation may be applied in any appropriate location on the scalp in order to modulate the activity of superficial cortical brain regions (e.g., the outer cortical regions of the brain) that would otherwise by undesirably modulated by the application of the transcranial magnetic stimulation. For example, the methods described herein may include placing a transcranial direct current electrode against the patient's scalp under at least one of the one or more transcranial magnetic stimulation electromagnets.

The step of applying transcranial magnetic stimulation to the deep brain target may include applying transcranial magnetic stimulation from the one or more transcranial magnetic stimulation electromagnets at a power sufficient to induce seizures without the application of the transcranial direct current stimulation modulating excitability of the cortical brain regions.

Also described herein is a method for applying transcranial magnetic stimulation to modulate a deep brain target while reducing or preventing seizures by modulating cortical brain regions, the method comprising: targeting a deep-brain target region with an array of transcranial magnetic stimulation electromagnets; applying transcranial direct current stimulation from the scalp to modulate excitability of cortical brain regions between the array of transcranial magnetic stimulation electromagnets and the deep-brain target; and applying transcranial magnetic stimulation to the deep brain target while modulating the cortical regions by the transcranial direct current stimulation

Also described herein are methods of applying transcranial magnetic stimulation to a patient to modulate a deep brain target while preventing or reducing seizures by modulating cortical brain regions, the method comprising: aiming at one or more transcranial magnetic stimulation electromagnets at a deep-brain target; positioning a transcranial direct current stimulation cathode electrode on the patient's scalp under the center of the transcranial magnetic stimulation electromagnet and placing a transcranial direct current stimulation anode electrode at a remote location on the scalp; applying transcranial direct current stimulation; and pulsing the one or more transcranial magnetic stimulation electromagnets at a rate and intensity to achieve a neuromodulation effect at the deep-brain target, whereby cortical brain regions superficial to the deep brain target are stabilized by the transcranial direct current stimulation such that the likelihood of seizures is reduced.

Another variation of a method of applying transcranial magnetic stimulation to a patient to modulate a deep brain target while preventing or reducing seizures by modulating cortical brain regions includes the steps of: aiming a plurality of transcranial magnetic stimulation electromagnets at the same deep brain target; positioning a plurality of transcranial direct current stimulation cathode electrodes on the patient's scalp under the centers of the transcranial magnetic stimulation electromagnets and positioning a common transcranial direct current stimulation anode electrode at a remote location on the patient's scalp; applying transcranial direct current stimulation; and pulsing the plurality of transcranial magnetic stimulation electromagnets at a rate and intensity to achieve a desired neuromodulation effect at the deep brain target, whereby cortical brain regions superficial to the deep brain target are stabilized by the transcranial direct current stimulation such that the likelihood of seizures is reduced.

In some variations, the number of transcranial direct current stimulation anode electrodes is matched to the number of transcranial direct current stimulation cathode electrodes.

Any of the methods described herein may be used to treat a disorder. For example, any of these methods may be used to treat a disorder selected from the group consisting of: pain, depression, addiction, Alzheimer's disease, attention deficit disorder, autism, anorgasmia, cerebral palsy, bipolar depression, unipolar depression, epilepsy, generalized anxiety disorder, acute head trauma, hedonism, obesity, Obsessive Compulsive Disorder, acute pain, chronic pain, Parkinson's disease, persistent vegetative state, phobia, post-traumatic stress disorder, post-stroke rehabilitation or regenesis, post-head trauma, social anxiety disorder, Tourette's Syndrome, hemorrhagic stroke, and ischemic stroke.

Also described herein are deep-brain transcranial magnetic stimulation controllers for applying transcranial magnetic stimulation to modulate a deep brain target while reducing side effects by modulating cortical brain regions, the controller comprising: a transcranial direct current stimulation activation output configured to regulate application of transcranial direct current stimulation by at least a first set of electrodes; a transcranial magnetic stimulation activation output configured to regulate application of transcranial magnetic stimulation by one or more transcranial magnetic stimulation electrodes; and controller logic configured to regulate the outputs so that transcranial magnetic stimulation is applied immediately after or concurrently with the application of transcranial direct current stimulation. The controller may be configured to regulate the outputs so that transcranial magnetic stimulation is activated concurrently with the activation of transcranial direct current stimulation.

In some variations, the controller includes a user input configured to trigger activation of transcranial magnetic stimulation and transcranial direct current stimulation.

The controller logic may be configured to prevent activation of transcranial magnetic stimulation unless transcranial direct current stimulation is activated. In some variations, the controller logic is configured to prevent activation of transcranial magnetic stimulation unless transcranial direct current stimulation has been activated within a predetermined time period. The predetermined time period may be approximately 5 minutes, approximately 10 minutes, approximately 20 minutes, approximately 30 minutes, etc.

Also described herein are deep-brain transcranial magnetic stimulation systems for applying transcranial magnetic stimulation to modulate a deep brain target while reducing side effects by modulating cortical brain regions, the system comprising: a plurality of transcranial magnetic stimulation electromagnets; one or more transcranial direct current electrode pairs; and a controller configured to synchronize the application of transcranial direct current stimulation and transcranial magnetic stimulation.

As mentioned above, the methods, device and systems described herein may combine deep-brain TMS and tDCS with tDCS applied to stabilize and mitigate seizure risks in the superficial cortex. Cortical neurons may be hyperpolarized and thus less excitable if they are located under the tDCS cathode. Thus, the tDCS cathode may be positioned on the scalp at the point where the magnetic field generated by the TMS electromagnet is greatest. In some variations, the system may include a frame or support frame to hold and/or position the TMS electromagnets relative to one or more tDCS electrodes. In some variations, the tDCS electrodes are integrated with the TMS electromagnets. For example, the tDCS electrodes may be connectable, coupled, or integral to a TMS electromagnet. The tDCS electrode may be part or connectable to the body of the TMS electromagnet, so that the TMS electromagnet may be targeted at a deep brain region while the tDCS electrode contacts the intervening scalp region, and thereby modulates the cortical region under the electrode.

In some variations, the tDCS cathode is a negative electrode, and the neurons under it may have their resting membrane potentials decreased, causing them to be hyperpolarized and thus less excitable. In this variations, the tDCS anode may be located remotely, e.g., on the opposite side of the head, but not underneath a TMS electromagnet. Because the tDCS anode is positive, the neurons under it may have their resting membrane potentials increased causing them to be partially depolarized and thus more excitable. Since the location of the anode is remote from the location where there is an intense magnetic field generated by a TMS electromagnet, hyperpolarization (and therefore a potential increase in sensitivity) is avoided even when applying TMS. Thus, the arrangement (and anodal/cathodal type current applied) may be correlated to the TMS electromagnet.

The application of tDCS and TMS as described herein may be performed so that both tDCS and TMS are delivered simultaneously. Both the tDCS and the TMS may be applied therapeutically in order to modulate a deep brain target region while simultaneously suppressing or reducing side effects such as seizures which may otherwise be elicited by the TMS.

In the application of the DC signal to the scalp, any appropriate electrode(s) may be used, particularly those that are compatible with concurrent operation of TMS. For example, electrodes may be sponge electrodes. In general, the larger the contact area of the electrode, the larger the area available for transfer of the tDCS signal. Larger contact areas may result in lower impedance at the electrode-scalp interface. Thus, any appropriate tDCS electrodes may be used.

A tDCS cathode may be positioned relative to the TMS electromagnet(s). For example, a tDCS electrode (e.g., cathode) may be positioned under each TMS coil center or behind each TMS coil center. For example, a cathode may be placed on the side of the coil which is distal with respect to the nearest anode. The placement and orientation of the TMS electromagnets may therefore determine the orientation of the tDCS electrodes. For example, the tDCS anode may be placed approximately 180 degrees around the head from the tDCS cathode, which may be placed relative to the TMS electromagnet.

In some variations, TMS and tDCS may be applied simultaneously. Thus, tDCS may provide protection against seizures generated by TMS. Although the TMS may be used to target deep brain target regions (nuclei, etc.), the tDCS may also be configured to convey some therapeutic effect as well. Since most clinical indications have multiple targets in a neural circuit, it is still possible that some therapy will be provided by tDCS.

### BRIEF DESCRIPTION OF THE DRAWINGS

FIG. 1 shows a simplified version of the arrangement of a TMS electromagnet and tDCS electrode pair arranged around a subject's head. Only a single TMS electromagnet is shown, though two or more TMS electromagnets (and two or more tDCS electrode or electrode pairs) may be used.

FIG. 2. illustrates a system having a plurality of TMS electromagnets and associated tDCS electrodes. In this example, the three TMS electromagnets are associated in combination with three cathodes and one common anode for tDCS. The TMS electromagnets are configured for deep-brain neuromodulation, and may all be oriented to a deep brain target (e.g., the Dorsal Anterior Cingulate Gyrus).

FIG. 3 is a simplified block diagram of one example of a controller for deep brain TMS including tDCS to reduce or eliminate side effects.

### DETAILED DESCRIPTION OF THE INVENTION

In general the deep brain TMS systems described herein include one or more TMS electromagnets that are configured for deep brain TMS and one or more pairs of tDCS electrodes configured to apply DC current to modulate cortical regions immediately adjacent (e.g., beneath) the TMS electromagnet(s) to reduce or eliminate side effects such as seizures. The systems described herein may include a controller configured to control the sequence and/or timing of the TMS stimulation and tDCS stimulation, so that the TMS stimulation occurs only after (or concurrent with) the start of tDCS stimulation.

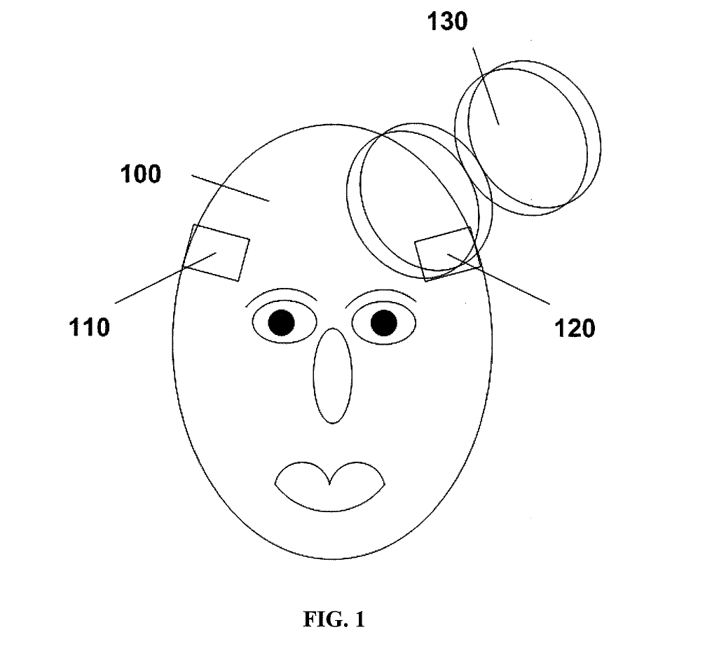

FIG. 1 illustrates one variation of a system showing a single TMS electromagnet 130 and single pair of tDCS electrodes 110, 120. This system may be configured for deep brain stimulation (e.g., stimulation of a target deep brain region) by including additional TMS electromagnets (not shown) or by moving the single TMS electromagnet so that it can stimulate the same target region from multiple positions around the outside of the subject's head.

In this example, tDCS cathode electrode 120 is centered under TMS electromagnet 130 on the scalp of the patient's head 100. Any of the tDCS electrodes described herein may be manufactured with a radial slot or other structure as known in the art to prevent strong eddy currents from being induced by TMS pulses. Such eddy currents can otherwise lead to electrode heating and scalp burns. Because of the potential for distortion of the magnetic field generated by TMS magnet 130 it is preferable to use non-ferromagnetic materials for the tDCS electrodes or non-metallic electrodes such as pads soaked with conductive fluid. The tDCS anode electrode 110 in this example is located at a position contralateral to the tDCS cathode electrode 120. The tDCS cathode electrode is positive and the tDCS anode electrode is positive. The presence of cathode electrode 120 may at least partially hyperpolarize the underlying cortex (e.g., by lowering its membrane potential and decreasing neural excitability). Thus, the application of the pulse electromagnetic field from TMS electromagnet 130 will be much less likely to trigger a seizure in the underlying cortex, when the TMS electromagnet is powered sufficiently to (alone or in combination with other TMS electromagnets) to modulate activity of a deep brain target region. The cortex underlying the tDCS anode electrode 110 may have its membrane potential increased, and may be partially polarized and therefore more excitable. In this example, this is not problematic in this configuration because the anode electrode is sufficiently distant from the TMS coils so that TMS-induced currents are relatively insignificant at that position, making it unlikely that TMS would trigger seizure activity at this position.

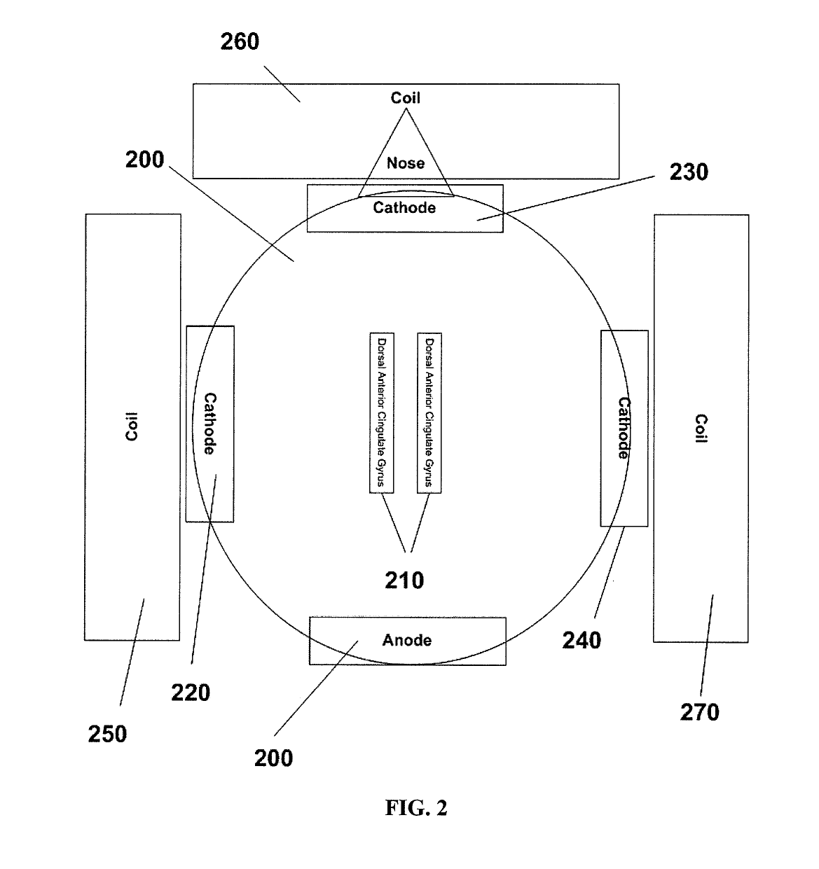

FIG. 2 demonstrates another example of a deep-brain TMS system configured to inhibit side effects by applying tDCS to the cortical region beneath the TMS electromagnets. In this configuration three TMS electromagnets 250, 260, 270 and three tDCS cathode electrodes 220, 230, 240 and a common tDCS anode electrode 200 are positioned around the subject's head. Although the simplified figure show the TMS electromagnets positioned approximately 90° apart around the head, the TMS electromagnets may be positioned closer to each other, and may indeed be positioned at an acute angle relative to each other (and out of the single plane shown in FIG. 2), while still focusing on a deep brain target so that the majority of the emitted TMS field reaches the deep brain target.

In FIG. 2, the deep brain target(s) have been identified as the Dorsal Anterior Cingulate Gyrus (DACG) regions 210 in the patient head 200. In this example, the tDCS cathode electrodes 220, 230, 240 are used in conjunction with a single common tDCS anode electrode 200 so that the cathodes may stabilize the cortex region underlying each TMS electromagnet (“coil” 250, 260 and 270). It is believed that the tDCS applied by the cathodes acts by decreasing the neural membrane potential and slightly hyperpolarizing the membrane, thus decreasing its excitability, thereby reducing the likelihood of a seizure or other side effects being inadvertently triggered by the TMS electromagnets 250, 260, 270 during deep-brain modulation of the DACG or other targets. By contrast, the cortex underlying tDCS anode electrode 220 may have its membrane potential increased and may therefore be partially polarized and made more excitable. Since there is no significant TMS stimulation at the cortical region underlying the anode, which is located distant from TMS electromagnets 250, 260, 270, there is little likelihood of triggering side effects such as seizures, from this region.

Any appropriate arrangement of TMS electromagnets and tDCS electrodes may be used. In general, the tDCS electrodes may be linked to the TMS electromagnets so that the tDCS electrodes may apply DC to modulate the cortical region underlying the TMS electrodes. As mentioned, it may be particularly useful to apply the tDCS to this cortical region (e.g., the region of cortex between the deep brain target and the TMS electromagnet) so that this cortical region is inhibited from triggering action potentials (e.g., by hyperpolarization) during the period of TMS.

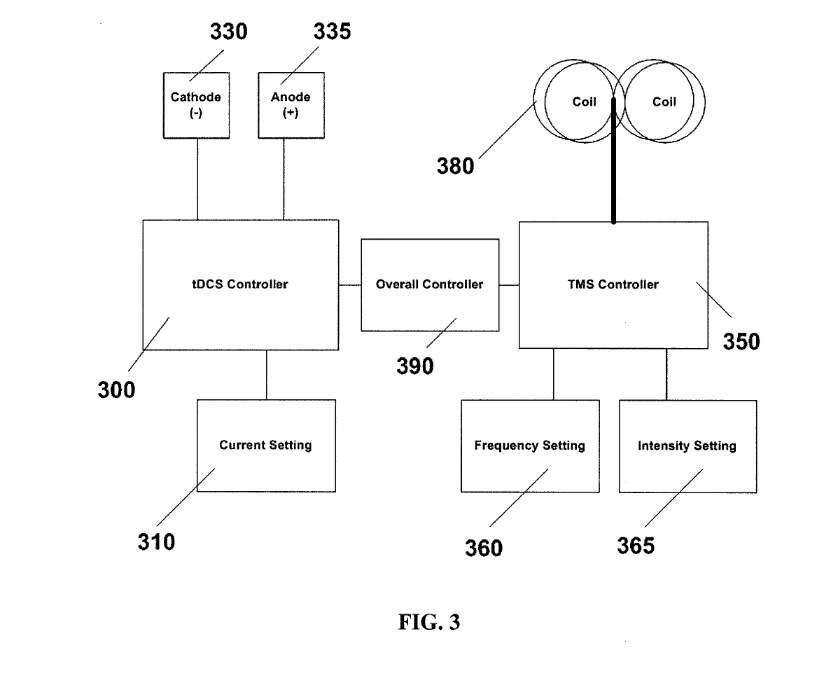

A block diagram of one variation of a deep-brain transcranial magnetic stimulation system for applying transcranial magnetic stimulation to modulate a deep brain target while reducing side effects by modulating cortical brain regions is shown in FIG. 3. In this example, the system includes a controller (“overall controller” 390) controlling both the tDCS electrodes 330, 335 and the TMS electromagnets 380 (although only one electromagnet is shown, more than one may be included). The controller may regulate the activity of the TMS electromagnet by including a TMS activation output that is configured to regulate application of transcranial magnetic stimulation by one or more transcranial magnetic stimulation electrodes. The TMS activation output may be part of the overall controller, or it may be part of a TMS controller 350. In some variations the TMS controller is integral to the overall controller 390. The controller may regulate the activity of the tDCS electrodes though a tDCS activation output that is configured to regulate application of transcranial direct current stimulation by the tDCS electrodes. The tDCS activation output may be part of the overall controller or part of a tDCS controller (which may be integral or separate from the overall controller). In general, the controller regulates the application of TMS and tDCS so that deep brain TMS is applied only after or concurrent with tDCS to the cortical region between the TMS electromagnets and the deep brain target. Thus, the controller may include controller logic (e.g., hardware, software, firmware, etc.) configured to regulate the outputs so that transcranial magnetic stimulation is applied immediately after or concurrently with the application of transcranial direct current stimulation.

In the exemplary system shown in FIG. 3, continuous neuromodulation by transcranial Direct Current Stimulation may be regulated by the controller; in this example, the controller includes a tDCS controller 300, an overall controller 390 and a TMS controller 350. The tDCS controller may control the activity of the tDCS electrodes, including the energy applied by the electrodes. For example, a tDCS controller may be connected to (or may include) a Current Setting 310 module that provides input and output to the tDCS cathode (negative) electrode 330 and anode (positive) electrode 335. In some variations, the tDCS controller may regulate the voltage/current applied (DC current) by limiting the applied current to prevent patient injury or discomfort. The current setting may be adjustable (e.g., user-defined), preset, or defined by the controller based on feedback from the patient or other portions of the system. Although only two tDCS electrodes are shown, any appropriate number may be used, as indicated previously.

The TMS portion of the system may be controlled in part by the TMS controller 350, as mentioned above. In FIG. 3, the TMS controller includes inputs determining the envelope of the applied energy. For example, the controller may receive input of frequency settings 360, intensity setting 365, and the like. The TMS controller may include or be connected to the energy source (e.g., driver) that provides the current to the TMS electromagnet(s) based on the signal timed and conditioned by the controller(s). In FIG. 3, the TMS Electromagnet is shown as a conventional flat-plane double coil, but other embodiments with other coil shapes are applicable and may be used.

The entire system (e.g., overall controller) may include a user input to trigger activation of the deep brain TMS. The system may be controlled by the controller so that deep brain TMS is only applied during or after the start of tDCS stimulation to the cortical regions through which the electromagnetic field of the TMS electromagnets substantially passes on the way to the deep brain target. Thus, the TMS neuromodulation of the deep brain target may occur substantially simultaneously with tDCS delivery on more cortical regions. In some variations, the system is set up so that the TMS occurs after the tDCS delivery, so that the underlying cortical regions have been modulated (or continue to be modulated) by the tDCS. Thus, the controller (which may include Overall controller 390) may control the switching of both the tDCS and TMS subsystems (e.g., TMS and tDCS Controllers).

As mentioned above, in some variation, a plurality of tDCS cathode electrodes along with one or a plurality of tDCS anode electrodes are controlled. Similarly, a plurality of TMS electromagnets may be controlled. In some variations multiple anodes may be used along with one or a plurality of tDCS cathode

In other embodiments of transcranial Direct Current Stimulation electrode placement the number of cathode electrodes is greater than the number of Transcranial Magnetic Stimulation electromagnets at positions in addition to locations under the centers of the TMS electromagnets or replacement of those centrally locations. Positioning of the tDCS cathode either: (1) under each TMS coil center or (2) behind each TMS coil center (cathode placed on the side of the coil which is distal with respect to the nearest anode) will determine the orientation of the tDCS electrodes; the tDCS anode will be placed in a location distant from the tDCS cathode. In some variations, the number of tDCS anode electrodes is greater than one when there are multiple tDCS cathode electrodes, rather than having a single common tDCS anode electrode.

The various embodiments described above are provided by way of illustration only and are not intended to be limiting to the embodiments described. Based on the above discussion and illustrations, those skilled in the art will readily recognize that various modifications and changes may be made to the present invention without strictly following the exemplary embodiments and applications illustrated and described herein. Such modifications and changes do not depart from the true spirit and scope of the present invention.

1. A method for applying transcranial magnetic stimulation to modulate a deep brain target while reducing side effects by modulating cortical brain regions, the method comprising:
targeting a deep-brain target region with one or more transcranial magnetic stimulation electromagnets;
applying transcranial direct current stimulation from the scalp to modulate excitability of cortical brain regions between the one or more transcranial magnetic stimulation electromagnets and the deep-brain target; and
applying transcranial magnetic stimulation to the deep brain target while modulating the cortical regions by the transcranial direct current stimulation.

2. The method of claim 1 wherein the step of applying transcranial magnetic stimulation comprises aiming a plurality of transcranial magnetic stimulation electromagnets at the deep brain target.

3. The method of claim 1, wherein the step of applying transcranial magnetic stimulation comprises aiming at least one transcranial magnetic stimulation electromagnet at the deep brain target while moving the transcranial magnetic stimulation electromagnet about the subject's head

4. The method of claim 1, wherein the step of applying transcranial magnetic stimulation to the deep brain target comprises applying energy to the transcranial magnetic stimulation electromagnets while concurrently applying the transcranial direct current stimulation from the scalp.

5. The method of claim 1, wherein the step of applying transcranial direct current stimulation comprise applying cathodal transcranial direct current stimulation to the region of the scalp between the one or more transcranial magnetic stimulation electromagnets and the deep-brain target.

6. The method of claim 1, wherein the step of applying transcranial direct current stimulation comprise applying anodal transcranial direct current stimulation to regions of the scalp remote from the TMS electromagnets.

7. The method of claim 1, wherein the steps of applying transcranial direct current stimulation and applying transcranial magnetic stimulation are simultaneously applied.

8. The method of claim 1, further comprising placing a transcranial direct current electrode against the patient's scalp under at least one of the one or more transcranial magnetic stimulation electromagnets.

9. The method of claim 1, wherein the step of applying transcranial magnetic stimulation to the deep brain target comprises applying transcranial magnetic stimulation from the one or more transcranial magnetic stimulation electromagnets at a power sufficient to induce seizures without the application of the transcranial direct current stimulation modulating excitability of the cortical brain regions.

10. A method for applying transcranial magnetic stimulation to modulate a deep brain target while reducing or preventing seizures by modulating cortical brain regions, the method comprising:
targeting a deep-brain target region with an array of transcranial magnetic stimulation electromagnets;
applying transcranial direct current stimulation from the scalp to modulate excitability of cortical brain regions between the array of transcranial magnetic stimulation electromagnets and the deep-brain target; and
applying transcranial magnetic stimulation to the deep brain target while modulating the cortical regions by the transcranial direct current stimulation

11. A method of applying transcranial magnetic stimulation to a patient to modulate a deep brain target while preventing or reducing seizures by modulating cortical brain regions, the method comprising:
aiming at one or more transcranial magnetic stimulation electromagnets at a deep-brain target;
positioning a transcranial direct current stimulation cathode electrode on the patient's scalp under the center of the transcranial magnetic stimulation electromagnet and placing a transcranial direct current stimulation anode electrode at a remote location on the scalp;
applying transcranial direct current stimulation; and
pulsing the one or more transcranial magnetic stimulation electromagnets at a rate and intensity to achieve a neuromodulation effect at the deep-brain target,
whereby cortical brain regions superficial to the deep brain target are stabilized by the transcranial direct current stimulation such that the likelihood of seizures is reduced.

12. A method of applying transcranial magnetic stimulation to a patient to modulate a deep brain target while preventing or reducing seizures by modulating cortical brain regions, the method comprising:
aiming a plurality of transcranial magnetic stimulation electromagnets at the same deep brain target;
positioning a plurality of transcranial direct current stimulation cathode electrodes on the patient's scalp under the centers of the transcranial magnetic stimulation electromagnets and positioning a common transcranial direct current stimulation anode electrode at a remote location on the patient's scalp;
applying transcranial direct current stimulation; and
pulsing the plurality of transcranial magnetic stimulation electromagnets at a rate and intensity to achieve a desired neuromodulation effect at the deep brain target,
whereby cortical brain regions superficial to the deep brain target are stabilized by the transcranial direct current stimulation such that the likelihood of seizures is reduced.

13. The method of claim 12, wherein the number of transcranial direct current stimulation anode electrodes is matched to the number of transcranial direct current stimulation cathode electrodes.

14. The method of claim 1 wherein the method is used to treat a disorder selected from the group consisting of: pain, depression, addiction, Alzheimer's disease, attention deficit disorder, autism, anorgasmia, cerebral palsy, bipolar depression, unipolar depression, epilepsy, generalized anxiety disorder, acute head trauma, hedonism, obesity, Obsessive Compulsive Disorder, acute pain, chronic pain, Parkinson's disease, persistent vegetative state, phobia, post-traumatic stress disorder, post-stroke rehabilitation or regenesis, post-head trauma, social anxiety disorder, Tourette's Syndrome, hemorrhagic stroke, and ischemic stroke.

15. A deep-brain transcranial magnetic stimulation controller for applying transcranial magnetic stimulation to modulate a deep brain target while reducing side effects by modulating cortical brain regions, the controller comprising:
a transcranial direct current stimulation activation output configured to regulate application of transcranial direct current stimulation by at least a first set of electrodes;
a transcranial magnetic stimulation activation output configured to regulate application of transcranial magnetic stimulation by one or more transcranial magnetic stimulation electrodes; and
controller logic configured to regulate the outputs so that transcranial magnetic stimulation is applied immediately after or concurrently with the application of transcranial direct current stimulation.

16. The controller of claim 15, wherein the controller is configured to regulate the outputs so that transcranial magnetic stimulation is activated concurrently with the activation of transcranial direct current stimulation.

17. The controller of claim 15, further comprising a user input configured to trigger activation of transcranial magnetic stimulation and transcranial direct current stimulation.

18. The controller of claim 15, wherein the controller logic is configured to prevent activation of transcranial magnetic stimulation unless transcranial direct current stimulation is activated.

19. The controller of claim 15, wherein the controller logic is configured to prevent activation of transcranial magnetic stimulation unless transcranial direct current stimulation has been activated within a predetermined time period.

20. The controller of claim 19, wherein the predetermined time period is selected from the group consisting of approximately 5 minutes, approximately 10 minutes, approximately 20 minutes, approximately 30 minutes.

21. A deep-brain transcranial magnetic stimulation system for applying transcranial magnetic stimulation to modulate a deep brain target while reducing side effects by modulating cortical brain regions, the system comprising:
a plurality of transcranial magnetic stimulation electromagnets;
one or more transcranial direct current electrode pairs;
a controller configured to synchronize the application of transcranial direct current stimulation and transcranial magnetic stimulation.
